(19) United States
(12) Patent Application Publication
(10) Pub. No.: US 2003/0195447 A1
(43) Pub. Date: Oct. 16, 2003

(54) Shoulder support device

(76) Inventors: Alexandra A. Cherubini, Brooklyn, NY (US); John Snowden, Byfield, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/123,868

(22) Filed: Apr. 16, 2002

(51) Int. Cl. A61F 5/00

(52) U.S. Cl. 602/20

(57) ABSTRACT

A shoulder supporter that retains the wearer's shoulder in a desired position is constructed so that it is more comfortable to wear and easier to put on and take off. The supporter has a first strap to extend about a shoulder and a second strap coupled to the first strap for anchoring at least a portion of the first strap in an anchoring location whereby the first and second straps cooperate to maintain the shoulder in the desired position. The second strap may be configured as a torso strap. The supporter may also have a release mechanism coupled to one of the straps and is operable from a side adjacent the front of the torso for releasing at least one of the straps. In this manner, the shoulder can move relatively unrestrained from the desired position. The supporter may also include elastic element adapted to exert a variable locating force on a shoulder such that, as the shoulder moves further away from the desired position, the force on the shoulder increases.

### FIELD OF THE INVENTION

The present invention relates to a support device and more particularly to a device for supporting a person's shoulders.

### BACKGROUND OF THE INVENTION

Shoulder supporters have been used as orthopedic devices for many years. Generally, such supporters have been used to hold a shoulder of an individual in a desired position after the individual has sustained a shoulder injury. Holding a shoulder in the desired position, for example in a position further backward, may aid in the healing process. Shoulder supporters may also be useful to maintain the user's shoulder in a certain posture during, for example, a sporting event such as horseback riding, or daily activities.

Shoulder supporters exist in the prior art that hold a person's shoulders in a desired position. Often these supporters comprise inextensible straps which extend across the front portion of each of a person's shoulders and meet with each other at a point adjacent the back of the wearer's torso. Fasteners and release mechanisms for these types of shoulder supports are often located adjacent the back of the wearer's torso.

### SUMMARY OF THE INVENTION

The supporter of the present invention is adapted to be worn such that it retains the shoulder in a desired position. The supporter is constructed so that it is comfortable to wear and easy to put on and remove. The supporter may include one or more features, each independently or in combination, contributing to increased comfort and ease of use.

In one embodiment, the shoulder supporter for maintaining a shoulder of a person in a desired position is disclosed. The supporter includes a first strap adapted to extend about a shoulder, the first strap having two ends. A second strap is coupled to each end of the first strap. At least a portion of the second strap is adapted to lie adjacent a back of the person's torso. The first and second straps together exerting a variable locating force against the shoulder to maintain the shoulder in the desired position. An elastic element cooperates with at least one of the first and second straps. The elastic element is adapted to exert a variable locating force on the shoulder, such that, as the shoulder moves further away from the desired position, the force on the shoulder increases.

In another embodiment, a shoulder supporter for maintaining shoulders of a person in desired positions is disclosed. The supporter includes a back panel adapted for placement adjacent a back of a person's torso, a first strap coupled to the back panel and adapted to extend about a first shoulder of the person; and a second strap coupled to the back panel and adapted to extend about a second shoulder of the person. The supporter also includes a torso strap coupled to the back panel. At least a first portion of the torso strap is adapted to be adjacent a back of the person's torso and at least a second portion of the torso strap is adapted to be adjacent a front of the person's torso. The supporter also includes an elastic element cooperating with at least one of the first, second and torso straps. The elastic element is adapted to exert a variable locating force on the shoulders such that, as the shoulders move further away from desired positions, the forces on the shoulders increase. The supporter also includes at least one release mechanism coupled to at least one of the first, second, and torso straps. The release mechanism is operable from a side adjacent a front of the person's torso for releasing at least one of the first, second, and torso straps, thereby allowing the at least one shoulder to move relatively unrestrained from a desired position.

A method for maintaining a shoulder of a person in a desired position is also disclosed. A method comprising donning at least a portion of a supporter about the shoulder. The supporter includes an elastic portion for exerting a variable force on the shoulder toward the desired position. The method also includes adjusting the supporter to provide an initial force exerted by the elastic portion to a desired amount to maintain the shoulder in a desired position.

Various embodiments of the present invention provide certain advantages and overcome certain drawbacks of prior shoulder supporters. Embodiments of the invention may not share the same advantages, and those that do may not share them under all circumstances. This being said, the present invention provides numerous advantages including the noted advantage of increased comfort and/or increased ease of use.

Further features and advantages of the present invention, as well as the structure of various embodiments, are described in detail below with reference to the accompanying drawings.

### BRIEF DESCRIPTION OF THE DRAWINGS

Various embodiments of the invention will now be described, by way of example, with reference to the accompanying drawings, in which:

FIG. 1 is a front view of a person wearing a shoulder supporter according to one embodiment of the invention;

FIG. 2 is a back view of a person wearing the shoulder supporter of FIG. 1;

FIG. 3 is a view of the shoulder supporter of FIGS. 1 and 2;

FIG. 4 is a side view of a person wearing the shoulder supporter of FIG. 1;

FIG. 5 is a back view of a person wearing another embodiment of a shoulder supporter; and

FIG. 6 is a back view of a person wearing yet another embodiment of a shoulder supporter.

### DETAILED DESCRIPTION

The supporter of the present invention is adapted to retain a wearer's shoulder in a desired position. Such a position may aid in the healing process of an injury, prevent the onset or reduce the likelihood of an injury, and/or improve the general posture of the wearer.

The supporter includes at least one shoulder strap that encircles the wearer's shoulder and a second strap which is connected to the shoulder strap and extends at least partially across the wearer's back. Together, these straps exert a rearward force on the shoulder to maintain the shoulder in the desired position. In one embodiment, the second strap may be configured as a torso strap that extends about the wearer's torso.

The shoulder supporter may be constructed and arranged to direct the wearer's shoulder toward the desired position by applying a variable locating force against the shoulder. This variable locating force is variable in direction and/or magnitude. The magnitude of the locating force may generally increase smoothly as a shoulder moves further away from the desired position. This may add to the comfort of the wearer by eliminating or reducing any sharp increase in force that might otherwise be applied against the shoulder. The variable locating force may also be preset by the wearer to provide an initial magnitude and/or direction thereby allowing the wearer to adjust the amount and type of support that will be provided. In one embodiment the support includes one or more elastic elements for applying the desired force.

The supporter is also constructed to be easy to put on or remove. In one embodiment, the supporter includes a release mechanism that is operable from the front side of the wearer's body. Thus, the user can readily put on or remove the supporter without undue manipulation, such as having to reach around their back to attach portions of the supporter, or requiring the assistance of another.

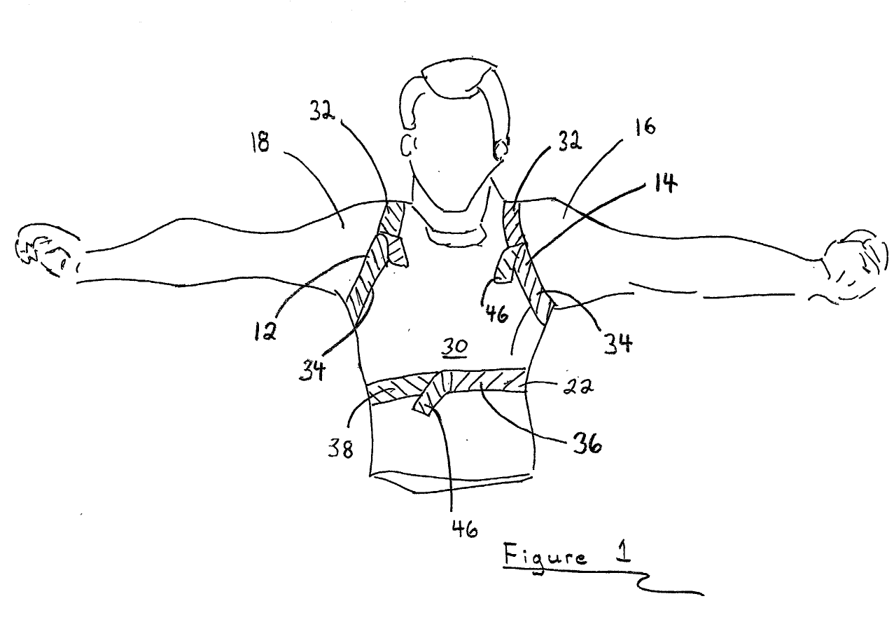

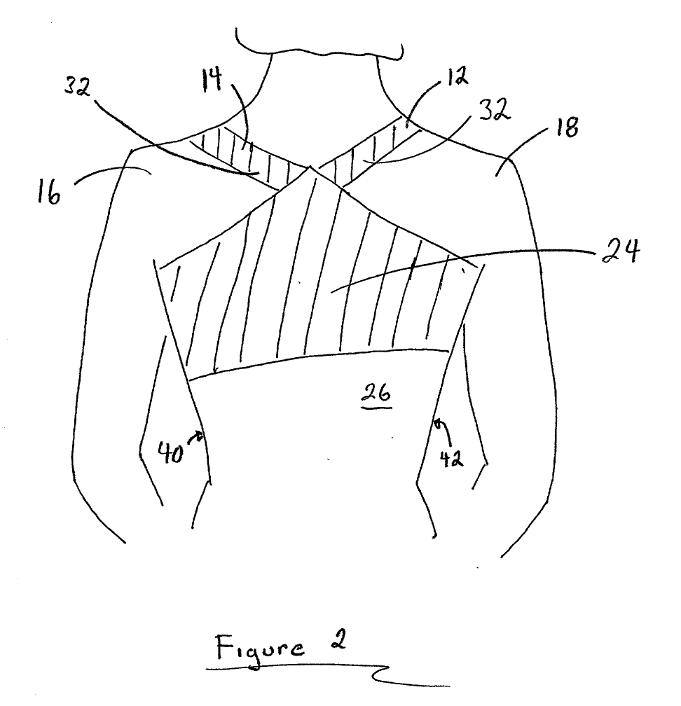

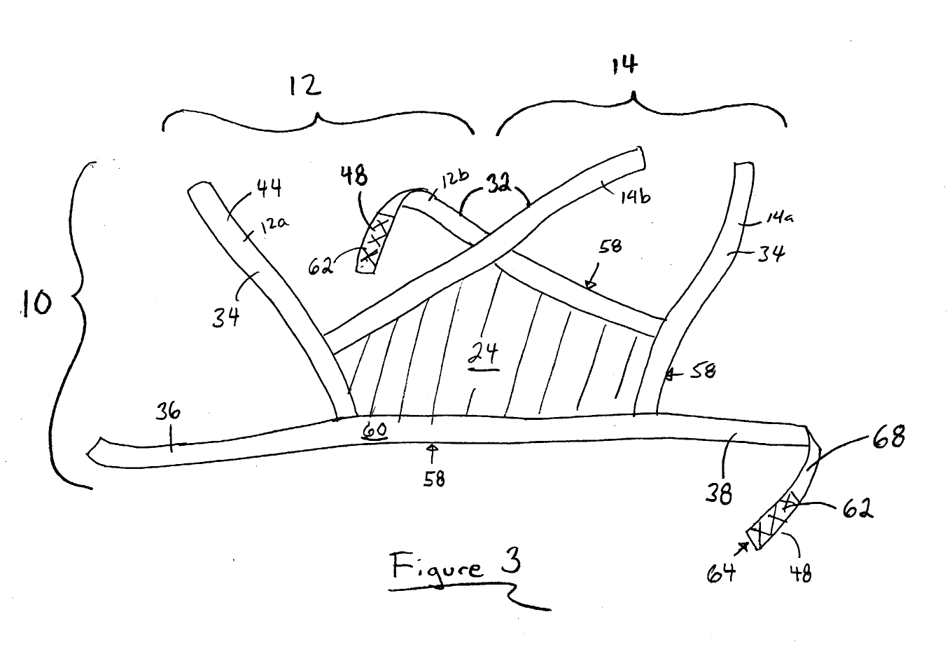

Turning now to the Figures and in particular FIGS. 1-3, the shoulder supporter 10 includes a first shoulder strap 12 and a second shoulder strap 14. Each of the first and second shoulder straps, has two ends, 12a, 12b and 14a, 14b, respectively. The first shoulder strap 12 is adapted to encircle a first shoulder 16 of a wearer. In a similar manner, the second shoulder strap 14 is adapted to encircle a second shoulder 18 of the wearer. In the embodiment shown (see FIGS. 2 and 3), the first and second shoulder straps 12, 14 are indirectly connected to each other.

In one embodiment, the supporter includes a torso strap 22 adapted to encircle the wearer's body. The torso strap 22 is also indirectly connected to the shoulder straps 12, 14 (see FIGS. 1, 2 and 3) and cooperates therewith to maintain the shoulder in the desired position 20. In one embodiment, each end 12a, 12b, 14a, 14b is coupled to the torso strap 22. It should be appreciated that the desired shoulder position may include a range of desired positions, as the present invention is not limited in this respect.

As shown in FIGS. 2 and 3, the supporter 10 may also include a back panel 24 adapted to cover a substantial portion of the wearer's back 26. As mentioned above, each of the straps are indirectly connected to each other. Thus, each of the straps 12, 14, 22 may be connected to the back panel 24. In one embodiment, each of the straps may be connected to the back panel at their respective ends 12a, 12b, 14a and 14b. Although the supporter of this embodiment includes a back panel, the present invention is not limited in this respect as no back panel 24 need be employed. In such an embodiment, each strap 12, 14 is directly connected to each other.

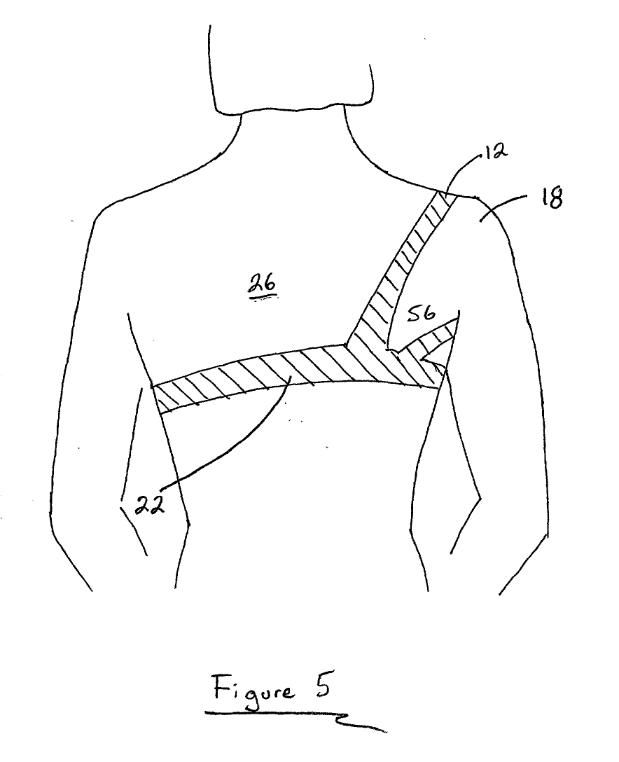

Further, although in the embodiment described with reference to FIGS. 1-3, three straps are employed, the present invention is not limited in this respect, as only two straps need be employed. For instance, in the embodiment shown in FIG. 5, the supporter 10 may only include a first strap 12 that encircles the wearer's right shoulder 18, and a torso strap 22 encircling the wearer's body, both straps 12, 22 cooperating to maintain the wearer's shoulder 18 in a desired position 20. In this embodiment, the torso strap anchors the shoulder strap in an anchoring location. Of course, the shoulder strap 12 may encircle a wearer's left shoulder 16 instead of the right shoulder 18.

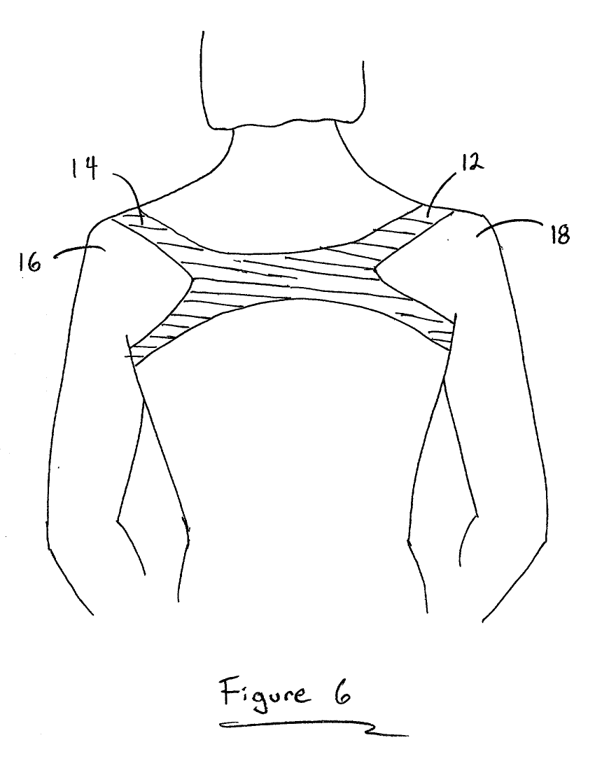

Also, as shown in FIG. 6, the supporter 10 may be configured with the first 12 and second 14 shoulder straps each adapted to encircle a respective first 16 and second shoulder 18. In this embodiment, both the first 16 and second shoulder 18 are maintained in a respective desired position 20 without the use of a torso strap 22 or a back panel 24, as the present invention is not limited to the use of a torso strap and/or a back panel.

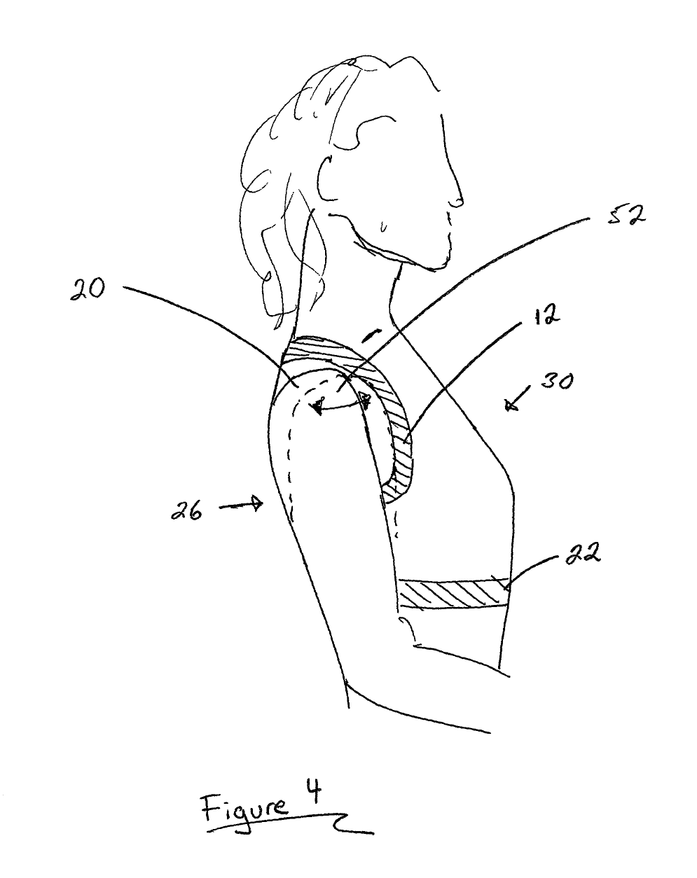

FIG. 4 depicts the supporter as it is directing a shoulder toward a desired position 20. In particular, the supporter, through one or more of the straps, exerts a force on the shoulder. The arrow shows the movement of the shoulder from a first position 52, to the desired position 20, which in this case is aft of the first position as the straps are exerting a rearward directed force on the shoulders. Other supporters may have a plurality of desired positions for each shoulder. In such cases, the shoulder may have more than one position that aids in the healing process of an injury, prevents the onset or likelihood of an injury, and/or improves the general posture of the wearer.

As discussed above, the supporter may include one or more release mechanisms 28. Such release mechanisms 28 may make the supporter easier to remove. To aid in accomplishing this, the release mechanism 28 is located at a position that can be reached from the front side 30 of the wearer, as shown in FIGS. 1 and 3. That is, in one embodiment, the release mechanism is located on a side of the supporter opposite the direction of the rearward directed force. Each of the shoulder straps 12, 14 of this embodiment comprises a first portion 32 which extends from the back panel 24 (if employed) over the top of the wearer's shoulder to connect with a second portion 34 which extends from the back panel 24 (if employed) beneath the wearer's arm and meets with the first portion 32 of the shoulder strap 12, 14 near a front facing or upward facing side of the shoulder. In the embodiment shown in FIGS. 1-3, the supporter 10 also includes release mechanism 28 on the torso strap 22. The torso strap 22 has a first portion 36 that extends about a first side 40 of the wearer and fastens to a second portion 38 of the strap that extends about an opposite side 42 of the wearer. The release mechanism 28 may also be near a position where a wearer could normally reach with their hand, thus making it more convenient to release.

The release mechanism may also function to fasten the supporter about the wearer. Because the release mechanism is located at a position where the wearer can easily grasp and manipulate the release mechanism, donning the supporter is also made easier.

To form the release mechanism, the first portion 32, 36 and the second portion 34, 38 of the straps 12, 14, 22 are connected with a hook and loop fastener 48 such as VELCRO@, although any suitable connectors may be used, such as buttons, snaps, hooks, zippers, buckles, or laces as the present invention is not limited in this respect. In the embodiment shown in FIGS. 1 and 3, the fastener 48 of each strap may be located at an intermediate point 44 of either strap portion creating an extension 46 that can be pulled by the user in a single motion to undo the fastener 48. However, the release mechanism 28 could alternatively be constructed without such an extension 46, as the invention is not limited in this respect.

The release mechanism 28 may also improve the adjustability of the supporter, allowing it to accommodate a wider range of wearer sizes. This may be accomplished with a fastener 48 that can be fastened at different positions on a strap 12, 14, 22. For instance, the hook and loop fastener on the first portion 32 of the shoulder straps 12, 14 in the embodiment shown in FIGS. 1-3 may be attached to the hook and loop fastener on the second portion 34 of the shoulder straps 12, 14 in different positions. This allows the shoulder straps to be adjusted to different sizes in order to accommodate different size wearers. As will be discussed below, adjusting the fastener to different positions may serve some additional functions.

The supporter 10 may have other adjustment features that do not also serve as release mechanisms 28. For instance, the junction between the back panel 24 and the torso strap on the ends 12a, 12b, 14a, or 14b of the shoulder straps may comprise an adjustable connection, such as a buckle or other suitable connection. Such a connection would allow the length of the strap to be adjusted to accommodate different sized wearers. Alternatively, the length of a strap 12, 14, 22 could be adjusted by positioning a portion of a strap over itself with the use of a buckle or other suitable adjuster to change the length of the strap.

As discussed above, according to another aspect of the invention, the shoulder supporter 10 may include one or more elastic elements 50 that apply a variable locating force against the shoulder 16, 18 to direct the wearer's shoulder toward the desired position 20. In one embodiment, the straps 12, 14, 22 are formed of an elastic material. The use of elastic materials allow the supporter to direct the shoulders toward the desired position 20 with a variable locating force that is variable in magnitude and/or direction. The nature of the elastic material also provides a variable locating force that gradually increases in magnitude as the shoulder departs from the desired position, thus eliminating any sharp increase in force that might otherwise be applied against the shoulder. While the straps of this embodiment are formed entirely of elastic material, the same effect can be accomplished with other suitable elastic elements located within the supporter, such as at the ends 12a, 12b, 14a, or 14b or in the middle of the straps as the invention is not limited in this respect. In one embodiment, the back panel 24 may also include an elastic element. In addition, one or more of the release mechanisms, such as the hook and loop fasteners 48, may also be formed, at least in part, of elastic material.

The adjustable nature of shoulder and torso straps 12, 14, 22 also allow the variable locating force to be preset by the wearer to a desired initial force magnitude and/or initial force direction. For example, the wearer may stretch any one or more of the straps 12, 14, 22 of the supporter a greater amount before fastening the fastener 48 if they desire a greater initial force magnitude, or alternatively may stretch the strap less if a lesser initial desired force magnitude is desired. The fastener 48 may also be fastened in different positions to adjust the initial direction of the variable locating force. For instance, the shoulder straps as shown in FIG. 2 may be placed in different initial positions about the shoulder 16, 18 to direct the shoulder in slightly different manners. Also, the torso strap 22 may be placed in a relatively low position (e.g., near a wearer's waist), as shown in FIG. 1, or a slightly higher position (e.g., near a wearer's sternum). The low position of the torso strap causes the variable locating force on the shoulder to be initially directed to a position that is lower than the initial variable locating force of the higher position.

The shoulder supporter 10 may be made entirely of elastic material, or alternatively they may only include elastic elements in particular areas. In this respect, one or more or all of the above-mentioned components forming the supporter may be formed of elastic material. For instance, the torso strap 22 may be formed of a relatively inextensible material such as some nylons, cottons or other suitable materials, while the shoulder strap is formed of an elastic material. Materials, such as cotton or mesh may serve to enhance the comfort provided to the user by allowing the skin beneath the supporter to breathe. The straps 12, 14, 22 may be connected to one another or to the back panel 24 by stitching, adhesives, or other suitable connection means. In some embodiments, such as those shown in FIGS. 5 and 6, the straps may be made from one continuous piece of material.

Embodiments of the supporter 10 may include substantially wide straps 12, 14, is 22, and, if used, a wide back panel 24. These wide surfaces may serve to reduce the amount of pressure applied to a given area of the body for a given force. This helps generally to improve the comfort afforded to the wearer by, among other things, reducing the amount of pressure points that may occur. The panel 24 and for one or more straps 12, 14, 22 of the support may also be padded, or made of padded material to help reduce pressure points between the supporter and the wearer. For instance, the back panel 24 may be made of a padded, elastic neoprene type material, it may include a gel material, or may include any other suitable cushiony material, as the present invention is not limited in this manner.

The supporter 16 may be put on and taken off in different manners. Some of these method are described hereafter; however, the methods described are only a few of the suitable methods that a wearer could use.

The supporter may be put on by pre-fastening both of the shoulder straps 12, 14 and then sliding each of the wearers arms 54 into the loops formed by the shoulder straps 12, 14. Once the shoulder straps 12, 14 are positioned appropriately, the torso strap 22 may be fastened and any necessary adjustments to the shoulder straps 12, 14 can be made by releasing their respective release mechanism 28 and adjusting the straps 12, 14 accordingly. Alternatively, the supporter may also be put on by placing the supporter near its intended position with all of the straps 12, 14, 22 in an unfastened state. The straps 12, 14, 22 may then be fastened one at a time until the supporter is put on in the appropriate position.

The supporter may be taken off by releasing the torso strap 22 alone and then sliding the wearer's arms out of the loops 56 formed by the shoulder straps 12, 14. Alternatively, one or both of the shoulder straps 12, 14 may be released along with the torso strap 22 for a quicker removal.

In embodiments where no release mechanism or torso strap are provided, the supporter may be put on by sliding the wearer's arm through the loop formed by one of the shoulder straps 12, 14 and then drawing the supporter up the wearer's arm 54 until it is in an appropriate position. The remaining portions of the supporter may then be pulled across the wearer's back 26. The wearer's other arm may then be strapped through the shoulder loop, with the shoulder strap about the wearer's shoulders. Supporters configured without a release mechanism may also be easily taken off by reversing the above process.

Any of the illustrated embodiments of the supporter lies generally close to the wearer's body when worn. This allows the supporter to be worn unobtrusively below a wearer's clothing if it is desired. The supporter can also be worn above a wearer's clothing.

The particular embodiment shown in FIG. 3 comprises a back panel 24 having five sides, the back panel being made of an elastic, neoprene type material and being black in color. The widest portion of the back panel being approximately 18 inches wide and the tallest portion being approximately 9 inches high. Each of the portions that comprise the torso strap 22 are made of an elastic material and are approximately 14 inches long in an unstretched state. One of the torso strap 36 portions has a loop material 60 covering its entire inner surface and the other portion has approximately 4 inches of hook material 62 disposed on the outer surface of its distal end 64. Each of the shoulder straps 12, 14 includes a first portion 32 extending approximately 13 inches in a generally upward direction from the outermost portion of the back panel 24. The entire inner surface 66 of each of these straps is lined with a loop material 60. The second portion 38 of each shoulder strap extends approximately 12 inches from the uppermost portion of the back panel 24. The outer surface 68 of the distal end 64 of each of these straps has approximately 4 inches of hook material 62 stitched thereon. Each of the torso 22 and shoulder straps 12, 14 are fixedly stitched to an edge of the back panel 24, which also serves to strengthen the back panel 24. Each of the straps 12, 14, 22 are approximately 1 inch wide.

Having described several embodiments of the invention in detail, various modifications and improvements will readily occur to those skilled in the art. Such modifications and improvements are intended to be within the spirit and scope of the invention. Accordingly, the foregoing description is by way of example only and is not intended as limiting. The invention is limited only as defined by the following claims and the equivalence thereto.

What is claimed is:

1. A shoulder supporter for maintaining a shoulder of a person in a desired position, the supporter comprising:
a first strap adapted to extend about a shoulder, the first strap having two ends;
a second strap coupled to each end of the first strap, at least a portion of the second strap being adapted to lie adjacent a back of the person's torso, the first and second straps together exerting a variable locating force against the shoulder to maintain the shoulder in the desired position; and
an elastic element cooperating with at least one of the first and second straps, the elastic element adapted to exert the variable locating force on the shoulder such that, as the shoulder moves further away from the desired position, the force on the shoulder increases.

2. The shoulder supporter of claim 1, wherein at least one of the first and second straps is elastic and forms the elastic element.

3. The shoulder supporter of claim 1, wherein each of the first and second straps is elastic and forms the elastic element.

4. The shoulder supporter of claim 1, wherein at least a portion of one of the first and the second straps is elastic and forms the elastic element.

5. The shoulder supporter of claim 1, wherein at least a portion of each of the first and second straps comprises the elastic element.

6. The shoulder supporter of claim 1, further comprising:
a release mechanism coupled to at least one of the first and second straps, the release mechanism operable from a side adjacent the front of the torso for releasing at least one of the first and second straps, thereby allowing the shoulder to move relatively unrestrained from the desired position.

7. The shoulder supporter of claim 6, wherein the release mechanism comprises a hook and loop fastener.

8. The shoulder supporter of claim 1, wherein the second strap comprises a torso strap, the torso strap having a first portion and a second portion, the first portion is adapted to be adjacent the back of the torso, the second portion is adapted to be adjacent the front of the torso.

9. The shoulder supporter of claim 7, wherein the torso strap is adapted to extend completely about the person's torso.

10. The shoulder supporter of claim 1, further comprising a back panel coupled to the first and second straps, the back panel adapted to be disposed adjacent a back of the person's torso.

11. The shoulder supporter of claim 10, wherein at least a portion of the back panel comprises an elastic element.

12. The shoulder supporter of claim 1, wherein the first strap is adapted to extend about a first shoulder and the second strap is adapted to extend about a second shoulder, whereby the first and second straps cooperate to maintain the shoulders in desired positions.

13. The shoulder supporter of claim 12, further comprising a torso strap coupled to the first and second straps, the torso strap having a first portion and a second portion, the first portion is adapted to be adjacent the back of the torso, the second portion is adapted to be adjacent the front of the torso.

14. A shoulder supporter for maintaining shoulders of a person in desired positions, the supporter comprising:
a back panel adapted for placement adjacent a back of a person's torso;
a first strap coupled to the back panel and adapted to extend about a first shoulder of the person;
a second strap coupled to the back panel and adapted to extend about a second shoulder of the person;
a torso strap coupled to the back panel, at least a first portion of the torso strap is adapted to be adjacent a back of the person's torso and at least a second portion of the torso strap is adapted to be adjacent a front of the person's torso;
an elastic element cooperating with at least one of the first, second and torso straps, the elastic element adapted to exert a variable locating force on the shoulders such that, as the shoulders move further away from desired positions, the forces on the shoulders increase; and
at least one release mechanism coupled to at least one of the first, second, and torso straps, the release mechanism operable from a side adjacent a front of the person's torso for releasing at least one of the first, second, and torso straps, thereby allowing the at least one shoulder to move relatively unrestrained from a desired position.

15. A method of maintaining a shoulder of a person in a desired position, the method comprising:
donning at least a portion of a supporter about the shoulder, the supporter having an elastic portion for exerting a variable force on the shoulder toward the desired position; and
adjusting the supporter to provide an initial force exerted by the elastic portion to a desired amount to maintain the shoulder in the desired position.

16. The method of claim 15, further comprising:
disengaging a release mechanism coupled to the supporter, the release mechanism operable from a front side of the person's torso for releasing the supporter, thereby allowing the shoulder to move relatively unrestrained from the desired position.
